(19) United States
(12) Patent Application Publication
(10) Pub. No.: US 2016/0250706 A1
(43) Pub. Date: Sep. 1, 2016

(54) WELDING SYSTEM PROVIDING REMOTE STORAGE OF VIDEO WELD DATA

(71) Applicant: Illinois Tool Works Inc., Glenview, IL (US)

(72) Inventors: Richard Beeson, Appleton, WI (US); William J. Becker, Manitowoc, WI (US)

(21) Appl. No.: 14/918,646

(22) Filed: Oct. 21, 2015

Related U.S. Application Data
(60) Provisional application No. 62/121,841, filed on Feb. 27, 2015.

(51) Int. Cl. B23K 9/095; A61F 9/06

(52) U.S. Cl. CPC B23K 9/095; A61F 9/064

(57) ABSTRACT

A system in accordance with an example implementation of this disclosure comprises welding headwear to be worn by a welder during a live welding operation, the headwear comprising: a camera operable to capture an image of a live welding operation; circuitry operable to analyze the captured image to determine a characteristic of the live welding operation, and associate the characteristic with the captured image; and memory operable to store the captured image and the associated characteristic for later retrieval. The headwear may comprise a communication interface operable to communicate with a remote server. The determined characteristic may comprise a welding parameter of a welding torch in the captured image. The determined characteristic may comprise a setting, or measured output, of welding equipment that powers and/or feeds wire to a torch being used in the live welding operation.

### PRIORITY CLAIM

This application claims priority to the following application(s), each of which is hereby incorporated herein by reference:

U.S. provisional patent application 62/121,841 titled “A WELDING SYSTEM PROVIDING REMOTE STORAGE OF VIDEO WELD DATA” filed on Feb. 27, 2015.

### BACKGROUND

Welding is a process that has increasingly become ubiquitous in all industries. While such processes may be automated in certain contexts, a large number of applications continue to exist for manual welding operations performed by skilled welding technicians. However, as the average age of the skilled welder rises, the future pool of qualified welders is diminishing. Furthermore, many inefficiencies plague the welding training process, potentially resulting in injecting a number of improperly trained students into the workforce, while discouraging other possible young welders from continuing their education. For instance, class demonstrations do not allow all students clear views of the welding process. Additionally, instructor feedback during student welds is often prohibited by environmental constraints.

### BRIEF SUMMARY

A system provides video data of a welding operation to a remote site. A welding helmet used in the welding operation contains a video display positioned so that a video presentation of the welding operation may be presented to the welder during the welding operation. A video camera is positioned in the helmet for generating raw unprocessed video of the welding operation, which is processed and presented on the display. A transmitter in the helmet transmits video to a remote site.

### BRIEF DESCRIPTION OF THE DRAWINGS

FIG. 1 shows an exemplary arc welding system in accordance with aspects of this disclosure.

FIG. 2 shows example welding equipment in accordance with aspects of this disclosure.

FIG. 3 shows example welding headwear in accordance with aspects of this disclosure.

FIG. 4 shows example circuitry of the headwear of FIG. 3.

FIGS. 5A-5C illustrate various parameters which may be determined from images of a weld in progress.

FIG. 6 is a flowchart illustrating a process for providing remote data storage of image data captured by welding headwear.

FIG. 7 shows an example image generated, presented and/or stored by the welding headwear of the system of FIG. 1

### DETAILED DESCRIPTION OF THE INVENTION

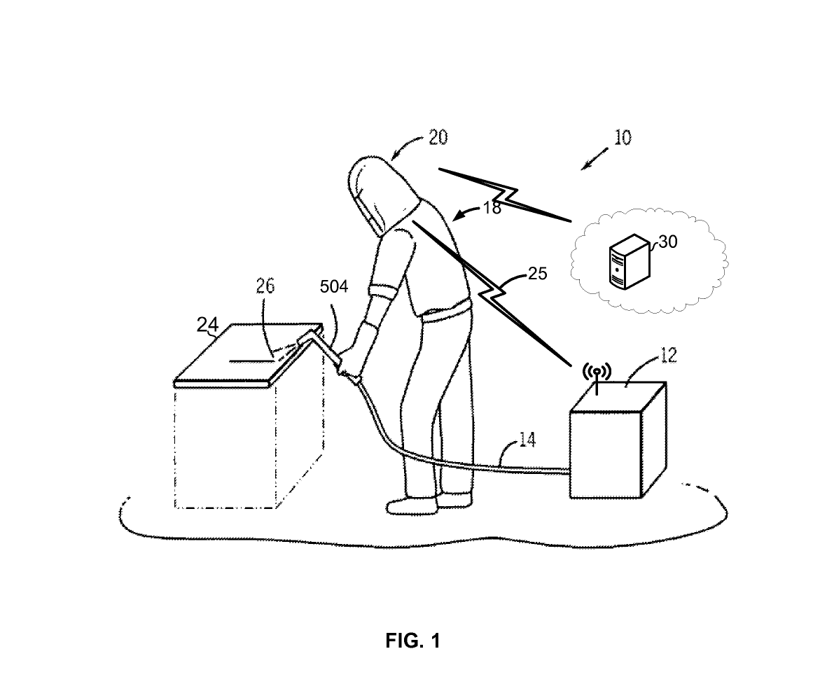

Referring to FIG. 1, there is shown an example welding system 10 in which an welder/operator 18 is wearing welding headwear 20 and welding a workpiece 24 using a torch 504 to which power or fuel is delivered by equipment 12 via a conduit 14 (for electrical welding conduit 15 provides the return path). The equipment 12 may comprise a power or fuel source, optionally a source of an inert shield gas and, where wire/filler material is to be provided automatically, a wire feeder. The welding system 10 of FIG. 1 may be configured to form a weld joint 512 by any known technique, including flame welding techniques such as oxy-fuel welding and electric welding techniques such shielded metal arc welding (i.e., stick welding), metal inert gas welding (MIG), flux cored arc welding (FCAW) tungsten inert gas welding (TIG), and resistance welding. TIG welding may involve no external filler metal or may involve manual, automated or semi-automated external metal filler.

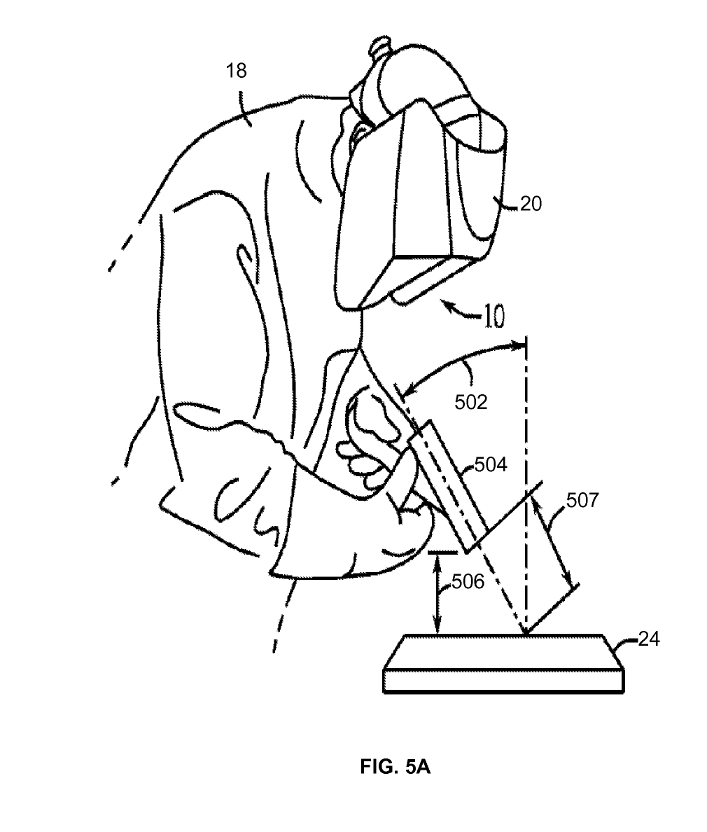

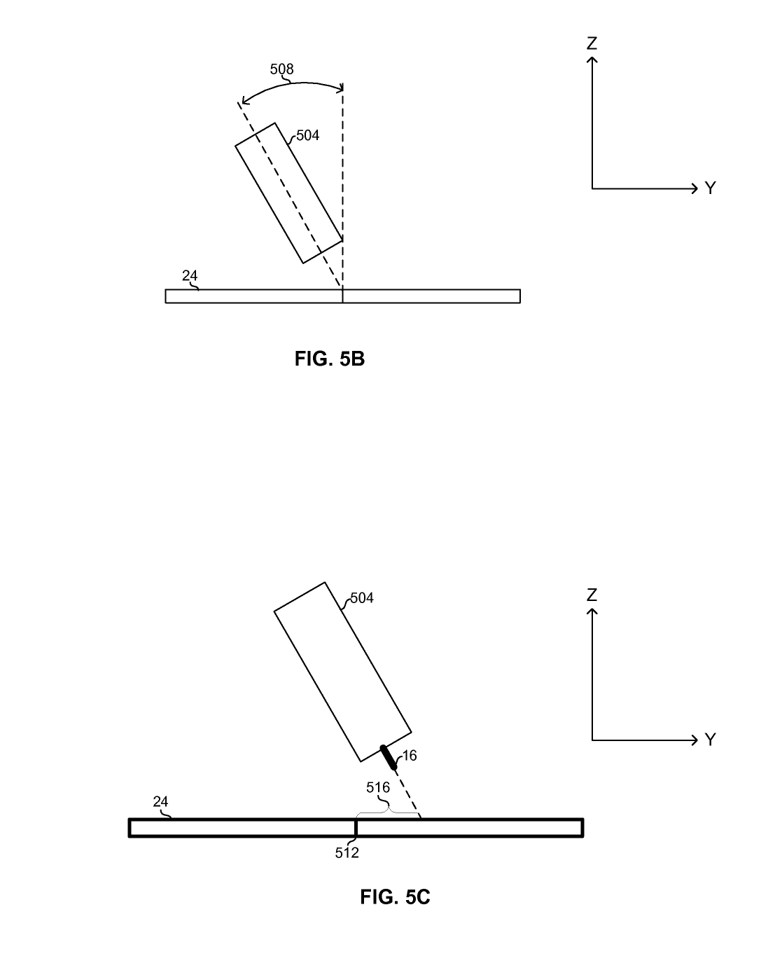

Optionally in any embodiment, the welding equipment 12 may be arc welding equipment that provides a direct current (DC) or alternating current (AC) to a consumable or non-consumable electrode 16 (better shown, for example, in FIG. 5C) of a torch 504, which may be a TIG torch, a MIG or flux cored torch (commonly called a MIG “gun”), or a stick electrode holder (commonly called a “stinger”). The electrode 16 delivers the current to the point of welding on the workpiece 24. In the welding system 10, the operator 18 controls the location and operation of the electrode 16 by manipulating the torch 504 and triggering the starting and stopping of the current flow. When current is flowing, an arc 26 is developed between the electrode and the workpiece 24. The conduit 14 and the electrode 16 thus deliver current and voltage sufficient to create the electric arc 26 between the electrode 16 and the workpiece. The arc 26 locally melts the workpiece 24 and welding wire or rod supplied to the weld joint 512 (the electrode 16 in the case of a consumable electrode or a separate wire or rod in the case of a non-consumable electrode) at the point of welding between electrode 16 and the workpiece 24, thereby forming a weld joint 512 when the metal cools.

As shown, and described more fully below, the equipment 12 and headwear 20 may communicate via a link 25 via which the headwear 20 may control settings of the equipment 12 and/or the equipment 12 may provide information about its settings to the headwear 20. Although a wireless link is shown, the link may be wireless, wired, or optical.

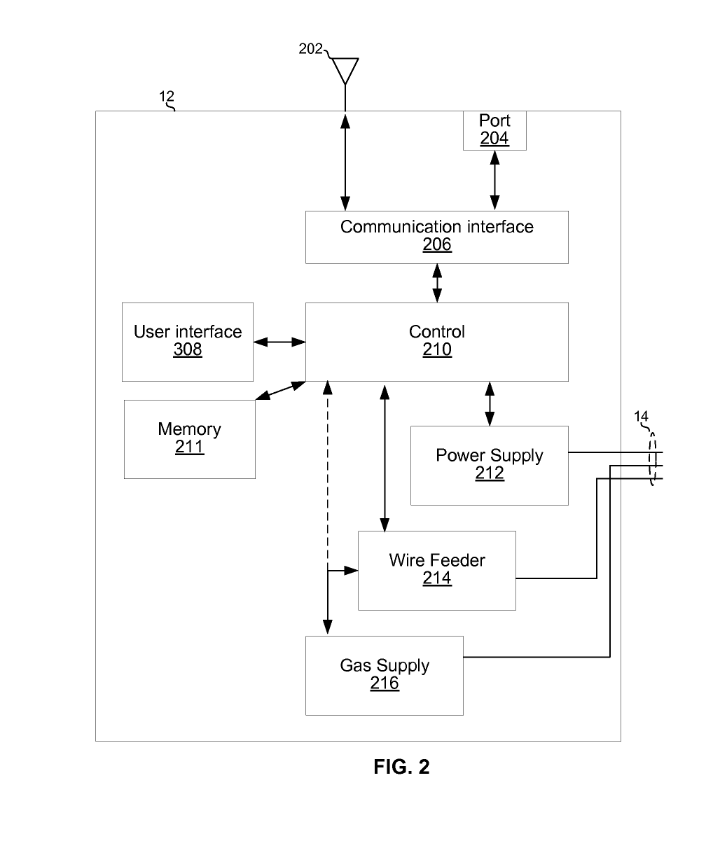

Referring to FIG. 2, equipment 12 comprises an antenna 202, a communication port 204, a communication interface circuitry 206, a user interface module 208, a control circuitry 210, a power supply circuitry 212, a wire feeder module 214, a gas supply module 216, and a memory 211.

Antenna 202 may be any type of antenna suited for the frequencies, power levels, etc., used by communication link 25.

Communication port 204 may comprise, for example, an Ethernet over twisted pair port, a USB port, an HDMI port, a passive optical network (PON) port, and/or any other suitable port for interfacing with a wired or optical cable.

Communication interface circuitry 206 is operable to interface control circuitry 210 to antenna 202 and/or port 204 for transmit and receive operations. For transmit operations, communication interface 206 receives data from control circuitry 210 and thereafter packetizes the data and converts the data to physical layer signals in accordance with protocols in use by communication link 25. For receive operations, communication interface 206 receives physical layer signals via antenna 202 or port 204 and thereafter recovers data from the received physical layer signals (demodulate, decode, etc.), and provides the data to control circuitry 210.

User interface 208 comprises electromechanical interface components (e.g., a screen, speakers, a microphone, buttons, a touchscreen, etc.) and associated drive circuitry. User interface 208 may generate electrical signals in response to user input (e.g., screen touches, button presses, control knob activations, mechanical switch activations, voice commands, etc.). User interface 208 includes driver circuitry to condition (e.g., amplify, digitize, etc.) the signals and send the conditioned signals to control circuitry 210. User interface 208 generates audible, visual, and/or tactile outputs (e.g., via speakers, a display, and/or motors/actuators/servos/etc.) in response to signals from control circuitry 210.

Control circuitry 210 may comprise a microcontroller and memory operable to process data from communication interface 206, user interface 208, power supply 212, wire feeder 214, and/or gas supply 216. Control circuitry 210 may output data and/or control signals to communication interface 206, user interface 208, power supply 212, wire feeder 214, and/or gas supply 216. Control circuitry 210 may store data in memory 211 or retrieve data from memory 211.

Power supply circuitry 212 comprises circuitry for generating power to be delivered to welding electrode 16 via conduit 14. Power supply circuitry 212 may comprise, for example, one or more voltage regulators, current regulators, inverters, and/or the like. The voltage and/or current output provided by power supply circuitry 212 may be controlled by a control signal from control circuitry 210. Power supply circuitry 212 may also comprise circuitry for reporting the present current value and/or voltage value to the control circuitry 210. In an example implementation, power supply circuitry 212 may comprise circuitry for measuring the voltage and/or current on conduit 14 (at either or both ends of conduit 14) such that reported voltage and/or current is actual and not simply an expected value based on calibration.

Wire feeder module 214 is configured to deliver a consumable wire electrode 16 to a weld joint, e.g., shown as reference numeral 512 in FIG. 5C. Wire feeder module 214 may comprise, for example, a spool for holding the wire, an actuator for pulling wire off the spool to deliver to the weld joint 512, and circuitry for controlling the rate at which the actuator delivers the wire. The actuator may be controlled based on a control signal from control circuitry 210. Wire feeder module 214 may also comprise circuitry for reporting the present wire speed and/or amount of wire remaining to control circuitry 210. In an example implementation, wire feeder module 214 may comprise circuitry and/or mechanical components for measuring the wire speed, such that reported speed is actual and not simply an expected value based on calibration.

The gas supply module 216 is configured to provide shielding gas via conduit 14 for use during the welding process. The gas supply module 216 may comprise an electrically controlled valve for controlling the rate of gas flow. The valve may be controlled by a control signal from control circuitry 210 (which may be routed through the wire feeder 214 or come directly from the control 210 as indicated by the dashed line). The gas supply module 216 may also comprise circuitry for reporting the present gas flow rate to the control circuitry 210. In an example implementation, the gas supply module 216 may comprise circuitry and/or mechanical components for measuring the gas flow rate such that reported flow rate is actual and not simply an expected value based on calibration.

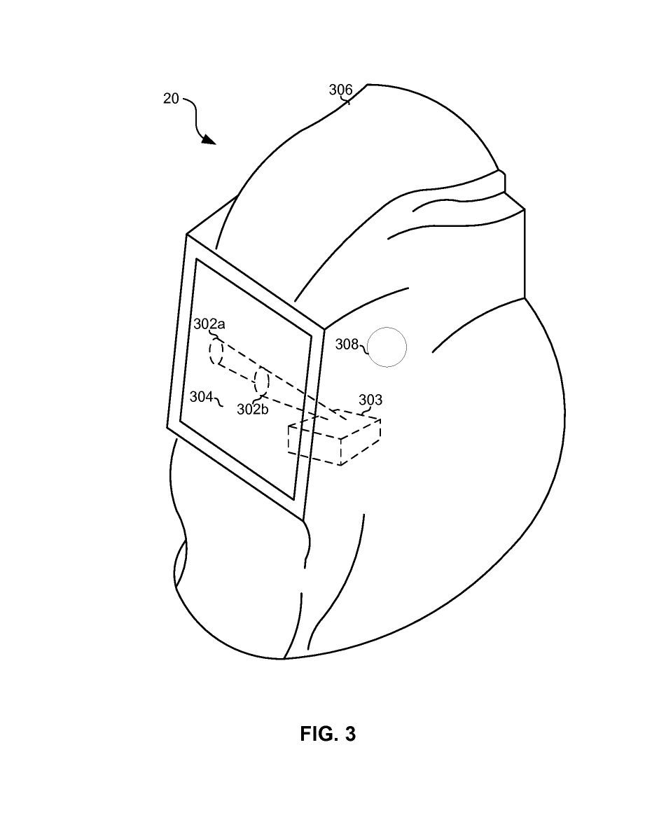

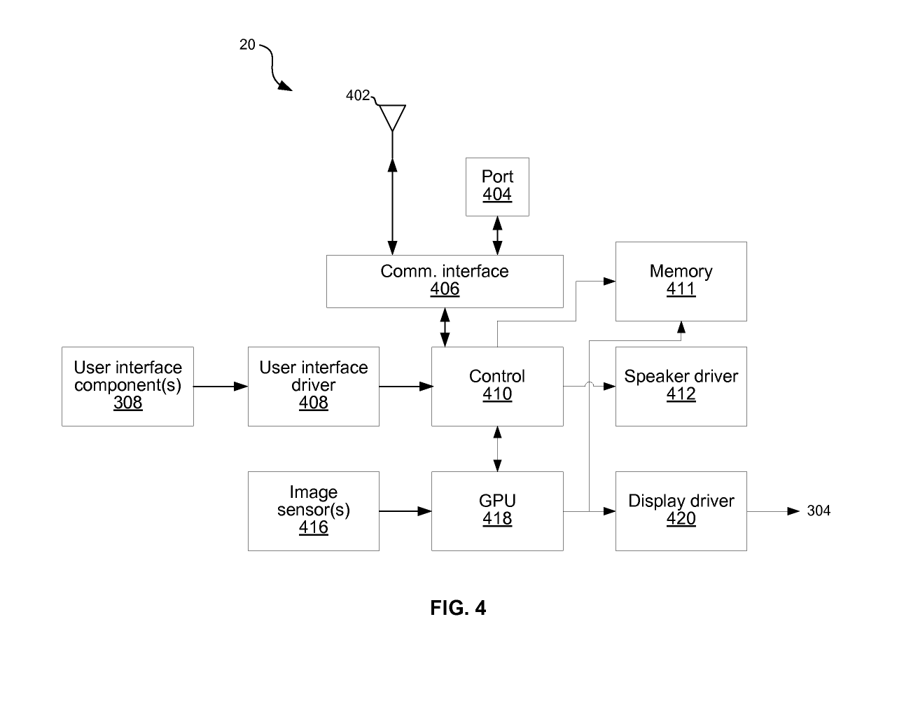

Referring to FIGS. 3 and 4, helmet 20 comprises a shell 306 in which are mounted: one or more cameras 303 comprising optical components 302a and 302b, image sensor(s) 416, a display 304, electromechanical user interface 308, an antenna 402, a communication port 404, a communication interface 406, a user interface driver 408, a central processing unit (CPU) control circuitry 410, speaker driver circuitry 412, a graphics processing unit (GPU) 418, display driver circuitry 420 and memory 411. In other embodiments, helmet 20 may take the form of a mask or goggles, for example.

Each of the camera's optical components 302a, 302b comprises, for example, one or more lenses, filters, and/or other optical components for capturing electromagnetic waves in the spectrum ranging from, for example, infrared to ultraviolet. Optical components 302a, 302b are for two cameras respectively and are positioned approximately centered with the eyes of a wearer of helmet 20 to capture stereoscopic images (at any suitable frame rate ranging from still photos to video at 30 fps, 100 fps, or higher) of the field of view the wearer of helmet 20 as if looking through a lens.

Display 304 may comprise, for example, a LCD, LED, OLED. E-ink, and/or any other suitable type of display operable to convert electrical signals into optical signals viewable by a wearer of helmet 20.

Electromechanical user interface 308 may comprise, for example, one or more touchscreen elements, speakers, microphones, physical buttons, switches, control knobs, etc. that generate electric signals in response to user input or user activation. For example, electromechanical user interface 308 may comprise capacitive, inductive, or resistive touchscreen sensors mounted on the back of display 304 (i.e., on the outside of helmet 20) that enable a wearer of helmet 20 to interact with user interface elements displayed on the front of display 304 (i.e., on the inside of helmet 20). In an example implementation, the optics 302, image sensors 416, and GPU 418 may operate as user interface components 308 by allowing a user to interact with the helmet 20 through, for example, hand gestures captured by the optics 302 and images sensors 416 and then interpreted by the GPU 418. For example, a gesture such as would be made to turn a knob clockwise may be interpreted to generate a first signal while a gesture such as would be made to turn a knob counterclockwise may be interpreted to generate a second signal.

Antenna 402 may be any type of antenna suited for the frequencies, power levels, etc. used by communication link 25.

Communication port 404 may comprise, for example, an Ethernet over twisted pair port, a USB port, an HDMI port, a passive optical network (PON) port, and/or any other suitable port for interfacing with a wired or optical cable.

Communication interface circuitry 406 is operable to interface control circuitry 410 to the antenna 402 and port 404 for transmit and receive operations. For transmit operations, communication interface 406 receives data from control circuitry 410, and packetizes the data and converts the data to physical layer signals in accordance with protocols in use by communication link 25. The data to be transmitted may comprise, for example, control signals for controlling equipment 12. For receive operations, communication interface 406 receives physical layer signals via antenna 402 or port 404, recovers data from the received physical layer signals (demodulate, decode, etc.), and provides the data to control circuitry 410. The received data may comprise, for example, indications of current settings and/or actual measured output of equipment 12 (e.g., voltage, amperage, and/or wire speed settings and/or measurements).

User interface driver circuitry 408 is operable to condition (e.g., amplify, digitize, etc.) signals from user interface 308.

Control circuitry 410 may comprise a microcontroller and memory operable to process data. Data may be processed from communication interface 406, user interface driver 408, and GPU 418, and to generate control and/or data signals to be output to speaker driver circuitry 412, GPU 418, and communication interface 406. Control circuitry 410 may store data in memory 211 or retrieve data from memory 211.

Signals output to communication interface 406 may comprise, for example, signals to control the settings of equipment 12. Such signals may be generated based on signals from GPU 418 and/or the user interface driver 408.

Signals from communication interface 406 comprise, for example, indications (received via antenna 402, for example) of current settings and/or actual measured output of equipment 12.

Speaker driver circuitry 412 is operable to condition (e.g., convert to analog, amplify, etc.) signals from control circuitry 410 for output to one or more speakers of user interface components 308. Such signals may, for example, carry audio to alert a wearer of helmet 20 that a welding parameter is out of tolerance, to provide audio instructions to the wearer of helmet 20, etc. For example, if the travel speed of the torch is determined to be too slow, such an alert may comprise a voice saying “too slow.”

Signals to GPU 418 comprise, for example, signals to control graphical elements of a user interface presented on display 304. Signals from the GPU 418 comprise, for example, information determined based on analysis of pixel data captured by images sensors 416. Image sensor(s) 416 may comprise, for example, CMOS or CCD image sensors operable to convert optical signals from cameras 303 to digital pixel data and output the pixel data to GPU 418.

Graphics processing unit (GPU) 418 is operable to receive and process pixel data (e.g., of stereoscopic or two-dimensional images) from image sensor(s) 416. GPU 418 outputs one or more signals to the control circuitry 410, and outputs pixel data to the display 304 via display driver 420. GPU 418 may also output unprocessed pixel data to memory 411 under control of control circuitry 410. Additionally, GPU 418 may also output processed pixel data to memory 411 under control of control circuitry 410.

The processing of pixel data by GPU 418 may comprise, for example, analyzing the pixel data, (e.g., a barcode, part number, time stamp, work order, etc.) to determine, in real time (e.g., with latency less than 100 milliseconds or, more preferably, less than 20 milliseconds, or more preferably still, 5 milliseconds), one or more of the following: name, size, part number, type of metal, or other characteristics of workpiece 24; name, size, part number, type of metal, or other characteristics of torch 504, electrode 16 and/or filler material; type or geometry of joint 512 to be welded; 2-D or 3-D positions of items (e.g., electrode, workpiece, etc.) in the captured field of view, one or more weld parameters (e.g., such as those described below with reference to FIGS. 5A, 5B and 5C) for an in-progress weld in the field of view; measurements of one or more items in the field of view (e.g., size of a joint or workpiece being welded, size of a bead formed during the weld, size of a weld puddle formed during the weld, and/or the like); and/or any other information which may be gleaned from the pixel data and which may be helpful in achieving a better weld, training the operator, calibrating the system 10, etc.

The information output from GPU 418 to control circuitry 410 may comprise the information determined from the pixel analysis. Such information may be stored in memory 411 by control circuitry 410.

The pixel data output from GPU 418 to display 304 may provide a mediated reality view for the wearer of helmet 20. In such a view, the wearer experiences a video presented on display 304 as if s/he is looking through a lens. The image may be enhanced and/or supplemented by an on-screen display. The enhancements (e.g., adjust contrast, brightness, saturation, sharpness, etc.) may enable the wearer of helmet 20 to see things s/he could not see with simply a lens. The on-screen display may comprise text, graphics, etc. overlaid on the video to provide visualizations of equipment settings received from control circuit 410 and/or visualizations of information determined from the analysis of the pixel data. The pixel data output from GPU 418 may be stored in memory 411 by control circuitry 410.

Display driver circuitry 420 is operable to generate control signals (e.g., bias and timing signals) for display 304 and to condition (e.g., level control synchronize, packetize, format, etc.) pixel data from GPU 418 for conveyance to display 304.

FIGS. 5A-5C illustrate various parameters which may be determined from images of a weld in progress. Coordinate axes are shown for reference. In FIG. 5A, the Z axis points to the top of the paper, the X axis points to the right, and the Y axis points into the paper. In FIGS. 5B and 5C, the Z axis points to the top of the paper, the Y axis points to the right, and the X axis points into the paper.

In FIGS. 5A-5C, equipment 12 comprises a MIG gun 504 that feeds a consumable electrode 16 to a weld joint 512 of workpiece 24. During the welding operation, a position of the MIG gun 504 may be defined by parameters including: contact-tip-to-work distance 506 or 507, a travel angle 502, a work angle 508, a travel speed 510, and aim.

Contact-tip-to-work distance may include a vertical distance 506 from a tip of torch 504 to workpiece 24 as illustrated in FIG. 5A. In other embodiments, the contact-tip-to-work distance may be a distance 507 from the tip of torch 504 to workpiece 24 at the angle of torch 504 to workpiece 24.

The travel angle 502 is the angle of gun 504 and/or electrode 16 along the axis of travel (X axis in the example shown in FIGS. 5A-5C).

A work angle 508 is the angle of gun 504 and/or electrode 16 perpendicular to the axis of travel (Y axis in the example shown in FIGS. 5A-5C).

The travel speed is the speed at which gun 504 and/or electrode 16 moves along the joint 512 being welded.

The aim is a measure of the position of electrode 16 with respect to the joint 512 to be welded. Aim may be measured, for example, as distance from the center of the joint 512 in a direction perpendicular to the direction of travel. FIG. 5C, for example, depicts an example aim measurement 516.

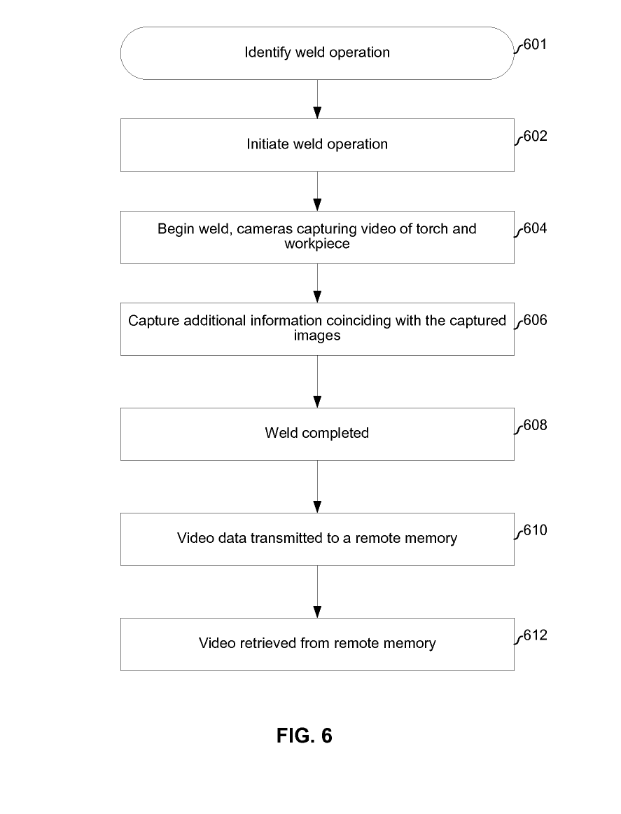

Referring to FIG. 6, a flowchart illustrates a process for welding a workpiece 24 while causing remote storage of image data based on such welding.

The process begins with block 601, in which one or more welds to be performed are determined by the headwear 20. The determination may be based on an identifier (e.g., a work order number, a part number, etc.) entered by the welder 18 through, for example, voice recognition and/or tactile input. Alternatively, or additionally, the welder 18 may view the workpiece to be welded from a distance and/or angle that permit the camera(s) 302 to capture an image of the workpiece from which an image processing algorithm can detect welds to be performed. For example, unique shapes, markings, and/or other features of a workpiece in the captured image view may be detected and used to retrieve an identifier associated with the workpiece.

In block 602, welder 18 initiates a welding operation. For example, welder 18 may give a voice command for welding system 10 to enter a weld mode, which voice command is responded to by user interface 308 of helmet 20. Control circuitry 410 configures the components of helmet 20 according to the voice command in order to display, on display 304, the live welding operation for viewing by the welder. The welder views the weld on display 304 and controls operation and positioning of electrode 16. Control circuitry 410 may respond to the voice command and send a signal to equipment 12 to trigger the weld mode in equipment 12. For example, control circuitry 210 disables a lock out so that power is delivered to electrode 16 via power supply 212 when a trigger on the torch is pulled by the welder. Wire feeder 214 and gas supply 216 may also be activated accordingly.

Block 602 thus represents the step of the welder placing the welding system in a weld mode so that the workpiece may be welded. Equipment 12 is configured by the welder 18 using user interface 208 based on the determined characteristics of the weld to be performed. For example, a constant current or constant voltage mode may be selected, a nominal voltage and/or nominal current may be set, a voltage limit and/or current limit may be set, and/or the like. Camera(s) 303 may be configured using electromechanical user interface 308. For example, expected brightness of the arc may be predicted (based on the equipment configuration and the characteristics of the weld to be made). The electric signals from user interface 308 may configure the darkness of a lens filter, for example.

In block 604, the operator begins welding. Workpiece 24 is placed into position, together with the electrode, relative to the field of view of camera lenses 302a, 302b. The trigger is activated by the welder, and a multimedia file is created/opened in memory and images of the weld operation begin to be captured by the camera 303 and stored to the multimedia file. The images may be stored as raw unprocessed pixel data coming from camera(s) 303. Alternatively (or additionally), the images may be stored as processed pixel data from GPU 418. In an example implementation, these events may be sequenced such that image capture starts first and allows a few frames during which the cameras 303 and/or display 304 are calibrated (adjusting focus, brightness, contrast, saturation, sharpness, etc.) before current begins flowing to the electrode, this may ensure sufficient image quality even at the very beginning of the welding operation. The multimedia file may be stored in memory 411 of helmet 20. Alternatively (or additionally), control circuitry 410 may transmit the images (unprocessed or processed) to the communication interface 406 for transmission to a remote memory such as memory 211 in equipment 12 and/or memory in server 30.

Still in block 604, in addition to storing the captured images, the images may be displayed in real-time on the display 304 and/or on one or more other remote displays to which the captured images are transmitted in real-time via link 25. In an example implementation, different amounts of image processing may be performed on one video stream output to the display 304 and another video stream output via link 25. In this regard, higher latency may be tolerable to the remote viewer such that additional processing may be performed on the images prior to presentation on the remote display.

In block 606, as the welding operation proceeds, the captured image data is processed and may be used to determine, in real-time (e.g., with latency less than 100 ms or, more preferably, less than 5 ms), present welding parameters such as those described above with reference to FIGS. 5A-5C. The determined welding parameters may be stored to memory along with the processed and/or unprocessed image data. For example, graphical representations of the welding parameters may be synchronized with the captured images and converted to text/graphics which are overlaid on the captured images prior to storing the images. Alternatively (or additionally), the determined welding parameters may be stored as metadata along with the captured image data.

Still referring to block 606, as the welding operation proceeds, settings and/or measured output of the equipment 12 may be received via link 25. Control circuitry 410 may adjust the settings based on the parameters determined. In this manner, equipment settings such as voltage, current, wire speed, and/or others may be adjusted in an attempt to compensate for deviations of the parameters from their ideal values. The equipment settings and/or measured output may be stored along with the captured image data. For example, the settings and/or measured output may be synchronized with the captured images and converted to text/graphics which are overlaid on the image data by GPU 418 prior to storing the image data and/or the identifier may be stored in metadata of the multimedia file in which the image data is stored.

Still referring to block 606, as the welding operation proceeds, other information may be captured (by the camera(s) 303 and/or other sensors) and stored along with the captured images. This other data may then be synchronized to the captured images and stored with the captured images (e.g., as metadata and/or converted to text/graphics and overlaid on the images). Such data may include, for example, an overall identifier of the weld operation determined in block 601, individual part numbers of the parts being welded (e.g., barcoded such that they can be automatically detected from the captured images), timestamps, climate (temperature, humidity, etc.), and/or the like. The multimedia file containing the may be indexed by any of this information for later searching and retrieval.

In block 608, the first weld operation on workpiece 24 is completed. In block 608 the multimedia file to which the images and other data were written during blocks 604 and 606 may be closed (e.g., file headers added, checksums calculated, etc.). In some instances, the file may be transferred for long term storage (e.g., from memory 411 of the helmet 20 to a database residing in memory of server 30).

Where the captured image data is stored as raw unprocessed pixel data, such raw unprocessed pixel data may be processed externally of helmet 20. In block 610, control circuitry 410 transmits the pixel data to, for example, a memory at server 30 via antenna 402 or port 404. A processor (not shown) at server 30 processes the raw unprocessed data and stores the processed data in memory at server 30. There may be more compute power at the server 30 and greater latency may be tolerated as compared to processing in helmet 20 prior to presentation on display 304. If there is too much latency inside the helmet, the welder may become disoriented. Similarly, pixel data already processed in helmet 20 under latency constraints (e.g., to condition it for real-time presentation on the display 304) may be further processed by the helmet 30 and/or by an external processor (such as in server 30). Such additional processing may enable determining additional and/or more-detailed information about the weld that there wasn't time and/or compute power to determine prior to real-time presentation of the captured images.

In block 612, the images captured during block 604 are transmitted from the memory of server 30 to a second remote location. For example, the images may be retrieved by an instructor or supervisor to review the work of a student or employee. As another example, the images may be reviewed by a quality control auditor as part of random quality inspections and/or as part of an investigation into a failed weld (e.g., if the welded part later fails in the field, the captured images and the information stored along with the images may be viewed to see if the weld process was the likely cause of the failure).

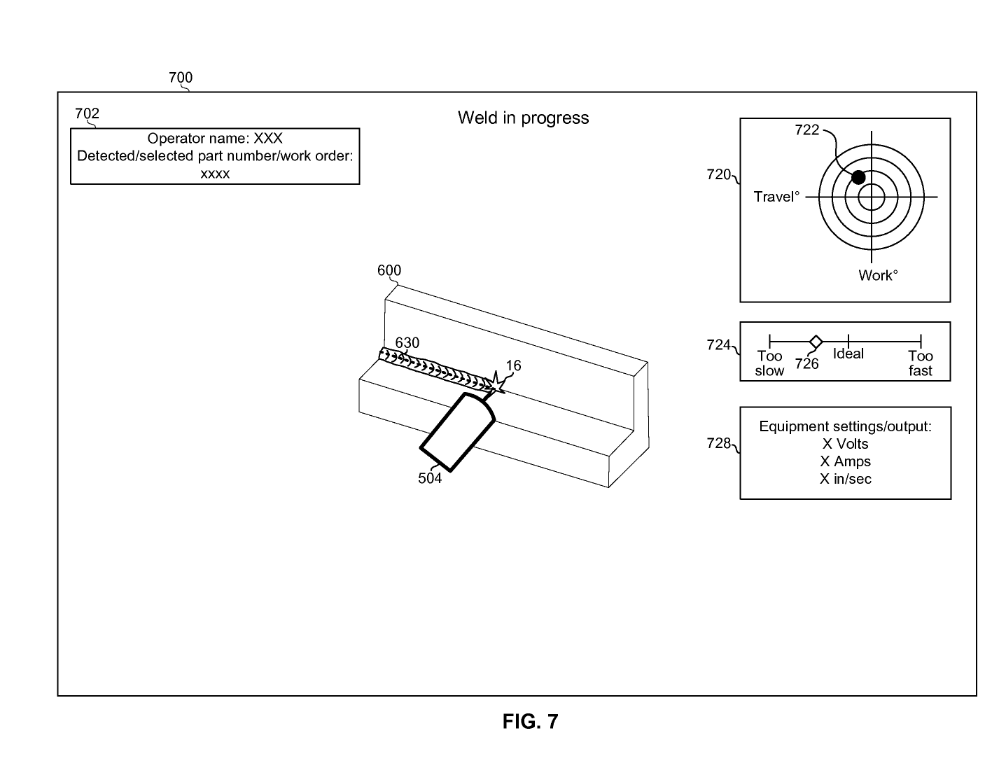

FIG. 7 depicts an example image presented and/or stored during a welding operation. Shown is a display 700 which represents the display 304 for in-helmet presentation of the image and represents a display external to the helmet 20 (e.g., of a computer that has retrieved the image from server 30) for presentation to a viewer other than a wearer of the helmet. The image comprises graphical elements 702, 720, 724, 728, and 730 overlaid on an image (e.g., one of many video frames) captured by the camera(s) 303. The overlaid graphics may be opaque or partially transparent. The graphic 702 (e.g., a text box) provides the viewer with information about the work being performed in the image (e.g., the part number of the workpiece, a work order number, and/or the like).

The graphics 720, 724, 728, and 730 present to the viewer one or more welding parameters measured during the weld being performed in the image. In the example shown, the graphic 720 comprises positional coordinate axes representing work angle and travel angle. The center of the coordinate system indicates the optimal orientation of the welding torch 504 during the weld. An actual orientation of the torch is indicated by dot 722. Other graphical representations of torch angle may be used instead of the “bull' s-eye” shown in FIG. 7. Some examples are described in United States Patent Application Publication 20090298024, which is hereby incorporated herein by reference. In the example shown, the graphic 724 comprises a graphical travel speed speedometer extending between a “too slow” marker and a “too fast” marker. A marker 726 indicating the actual travel speed is provided on the graphical speedometer. Other graphical representations of travel may be used instead of the linear speedometer shown in FIG. 7. Some examples are described in United States Patent Application Publication 20090298024, which is hereby incorporated herein by reference. The graphic 728 presents settings and/or actual measured output of the welding equipment 12 during the weld shown in the image. The graphic 730 shows the path traveled by the torch 504 up that point in the weld (i.e., historical aim of the torch 504).

A system in accordance with an example implementation of this disclosure, comprises welding headwear (e.g., 20) to be worn by a welder (e.g., 18) during a live welding operation, the headwear comprising: a video camera (e.g., 303) operable to capture an image of a live welding operation; circuitry (e.g., 410 and 418) operable to analyze the captured image to determine a characteristic of the live welding operation, and associate the characteristic with the captured image; and memory (e.g., 411 and/or memory of server 30) operable to store the captured image and the associated characteristic for later retrieval. The headwear may comprise a communication interface operable to communicate with a remote server (e.g., 30). The determined characteristic may comprises a welding parameter of a welding torch in the captured image. The determined characteristic may comprise a setting, or measured output, of welding equipment that powers and/or feeds wire to a torch being used in the live welding operation, where the setting is received via a communication link between the welding equipment and the welding headwear. The determined characteristics may comprise a work order number associated with the live welding operation, an identification of a welder performing the live welding operation, and/or a part number of a workpiece appearing in the captured image. The circuitry may operable to associate the determined characteristics with the captured image by generating a graphic indicative of the characteristic (e.g., 702, 720, 742, 728), and overlaying the graphic on the captured image (e.g., as shown in FIG. 7) prior to storage of the captured image to memory. The circuitry may operable to associate the determined characteristics with the captured image by writing the characteristics to metadata of a multimedia file containing the captured image. The circuitry is operable to, in response to a detection of a possible failure of a weld formed during the live welding operation (e.g., in response to a user entering an identifier associated with the live welding operation or analyzing a post-weld image of a workpiece welded during the live welding operation), retrieve the captured image and the associated characteristic from the memory.

The present methods and systems may be realized in hardware, software, or a combination of hardware and software. The present methods and/or systems may be realized in a centralized fashion in at least one computing system, or in a distributed fashion where different elements are spread across several interconnected computing systems. Any kind of computing system or other apparatus adapted for carrying out the methods described herein is suited. A typical combination of hardware and software may include a general-purpose computing system with a program or other code that, when being loaded and executed, controls the computing system such that it carries out the methods described herein. Another typical implementation may comprise an application specific integrated circuit or chip. Some implementations may comprise a non-transitory machine-readable (e.g., computer readable) medium (e.g., FLASH drive, optical disk, magnetic storage disk, or the like) having stored thereon one or more lines of code executable by a machine, thereby causing the machine to perform processes as described herein.

While the present method and/or system has been described with reference to certain implementations, it will be understood by those skilled in the art that various changes may be made and equivalents may be substituted without departing from the scope of the present method and/or system. In addition, many modifications may be made to adapt a particular situation or material to the teachings of the present disclosure without departing from its scope. Therefore, it is intended that the present method and/or system not be limited to the particular implementations disclosed, but that the present method and/or system will include all implementations falling within the scope of the appended claims.

As utilized herein the terms “circuits” and “circuitry” refer to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first set of one or more lines of code and may comprise a second “circuit” when executing a second set of one or more lines of code. As utilized herein, “and/or” means any one or more of the items in the list joined by “and/or”. As an example, “x and/or y” means any element of the three-element set {(x), (y), (x, y)}. In other words, “x and/or y” means “one or both of x and y”. As another example, “x, y, and/or z” means any element of the seven-element set {(x), (y), (z), (x, y), (x, z), (y, z), (x, y, z)}. In other words, “x, y and/or z” means “one or more of x, y and z”. As utilized herein, the term “exemplary” means serving as a non-limiting example, instance, or illustration. As utilized herein, the terms “e.g. and for example” set off lists of one or more non-limiting examples, instances, or illustrations. As utilized herein, circuitry is “operable” to perform a function whenever the circuitry comprises the necessary hardware and code (if any is necessary) to perform the function, regardless of whether performance of the function is disabled or not enabled (e.g., by a user-configurable setting, factory trim, etc.).

1. A system comprising:
welding headwear to be worn by a welder during a live welding operation;
a camera disposed in said welding headwear and operable to capture an image of said live welding operation;
circuitry operable to analyze said captured image to determine a characteristic of said live welding operation, and associate said characteristic with said captured image;
memory operable to store said captured image and said associated characteristic for later retrieval.

2. The system of claim 1, comprising a communication interface disposed in said welding headwear, said communication interface operable to communicate with a remote server.

3. The system of claim 1, wherein said characteristic comprises a welding parameter of a welding torch in said captured image.

4. The system of claim 1, wherein said welding parameter comprises one of:
a work angle of said welding torch, a travel angle of said welding torch, an aim of said welding torch, travel speed of said welding torch, and contact-tip-to-work distance.

5. The system of claim 1, wherein:
said characteristic comprises a setting of welding equipment that powers a torch being used in said live welding operation; and
said circuitry is operable to receive said setting via a communication link between said welding equipment and said welding headwear.

6. The system of claim 5, wherein said setting of said welding equipment comprises one of: a voltage setting, an amperage setting, and a wire speed setting.

7. The system of claim 1, wherein:
said characteristic comprises a measured output of welding equipment that powers a torch being used in said live welding operation; and
said circuitry is operable to receive said measured output via a communication link between said welding equipment and said welding headwear.

8. The system of claim 7, wherein said measured output of said welding equipment comprises one of: a voltage output and an amperage output.

9. The system of claim 1, wherein said characteristic comprises one of:
a work order number associated with said live welding operation,
an identification of a welder performing said live welding operation, and
a part number of a workpiece appearing in said captured image.

10. The system of claim 1, wherein, for said association of said characteristic with said captured image, said circuitry is operable to:
generate a graphic indicative of said characteristic; and
overlay said graphic on said captured image prior to storage of said captured image to memory.

11. The system of claim 1, wherein, for said association of said characteristic with said captured image, said circuitry is operable to store said associated characteristic in metadata of a multimedia file containing said captured image.

12. The system of claim 1, wherein said circuitry is operable to, in response to a detection of a possible failure of a weld formed during said live welding operation, retrieve said captured image and said associated characteristic from said memory.

13. A method comprising:
capturing, by a video camera disposed in welding headwear, an image of said live welding operation;
analyzing, by circuitry disposed in said welding headwear, said captured image to determine a characteristic of said live welding operation;
associating, by said circuitry disposed in said welding headwear, said determined characteristic with said captured image; and
storing, to memory disposed in said welding headwear, said captured image and said associated characteristic.

14. The method of claim 13, wherein said characteristic comprises a welding parameter of a welding torch in said captured image.

15. The method of claim 14, wherein said welding parameter comprises one of: a work angle of said welding torch, a travel angle of said welding torch, an aim of said welding torch, a travel speed of said welding torch, and contact-tip-to-work distance.

16. The method of claim 13, wherein:
said characteristic comprises a setting of welding equipment that powers a torch being used in said live welding operation; and
the method comprises receiving, via a communication interface disposed in said welding headwear, said setting via a communication link between said welding equipment and said welding headwear.

17. The method of claim 13, wherein:
said characteristic comprises a measured output of welding equipment that powers a torch being used in said live welding operation; and
the method comprises receiving, via a communication interface disposed in said welding headwear, said measured output via a communication link between said welding equipment and said welding headwear.

18. The method of claim 13, wherein said characteristic comprises one of:
a work order number associated with said live welding operation,
an identification of a welder performing said live welding operation, and
a part number of a workpiece appearing in said captured image.

19. The system of claim 13, wherein said associating said characteristic with said captured image comprises:
generating a graphic indicative of said characteristic; and
overlaying said graphic on said captured image prior to storing said captured image to memory.

20. The method of claim 13, comprising:
in response to detecting a possible failure of a weld formed during said live welding operation:
retrieving said captured image from memory; and
presenting said captured image for viewing.
